(19) United States
(12) Patent Application Publication
(10) Pub. No.: US 2019/0126198 A1
(43) Pub. Date: May 2, 2019

(54) POLLUTION CONTROL USING OZONE

(71) Applicant: University of Copenhagen, Copenhagen K (DK)

(72) Inventors: Matthew Stanley Johnsson, Lund (SE); Andreww Charles Butcher, Brøshøj (DK); Carl Meusinger, København N (DK); Kristoffer Skovlund Kipinen, København S (DK)

(73) Assignee: University of Copenhagen, Copenhagen K (DK)

(21) Appl. No.: 16/309,131

(22) PCT Filed: Jun. 16, 2017
(86) PCT No.: PCT/EP2017/064787

§ 371 Date: Dec. 12, 2018

(30) Foreign Application Priority Data
Jun. 17, 2016 (EP) 16175016.1

(51) Int. Cl. B01D 53/76; B01D 53/52; B01D 53/14; A61L 9/015

(52) U.S. Cl. CPC B01D 53/76; B01D 53/52; B01D 2251/104; A61L 9/015; B01D 53/1431

(57) ABSTRACT

This invention relates to a method for cleaning air comprising one or more pollutants, the method comprising contacting the air with thermal decompositions products of ozone.

### FIELD OF THE INVENTION

The present invention relates to a method for cleaning pollution from exhaust gas wherein the exhaust gas to be cleaned is subjected to a chemical and physical treatment.

### BACKGROUND OF THE INVENTION

Air pollution, such as odorous emissions, is produced by many industrial sources including biogas production, metal forging, livestock production, and food processing plants. Without emissions controls, pollution and odor are emitted as exhaust gas directly in the environmental vicinity of the source. Many odor problems are caused by sulfur-containing compounds, but depending on the source of pollution a vast number of compounds may be emitted. Apart from nuisance, substances may also cause health and/or environmentally damaging effects. It is presently not practical to measure smell in an objective physical or chemical manner, in part due to both the complex mixture of compounds and the nature of human sensation, so olfactometry is used, where the subjective perception of smell is characterized by a panel of well-trained individuals.

Odor problems and other airborne pollution represent a major obstacle to planning of new industrial facilities and to growth of existing production facilities in or in the proximity of residential areas, and are causes of negative publicity and unsatisfactory relations between industry and the local community. The nuisance caused by industrially derived odorous compounds and pollution has triggered much technological development. Presently a number of solutions to industrially derived odor problems are known but they are often either inefficient or expensive.

Typical output volumes of exhaust gascan be from 100 m³/hour from biogas cleaning/upgrading facilities to more than 10,000 m³/hour from pig farms. A typical sulfur-containing pollutant is H₂S, which can be found in such exhaust gas in concentrations ranging from typically 100 ppb to more than 1000 ppm, but has a smell threshold of 0.7 ppb.

Dilution using stacks or chimneys is a simple method but it entails high construction costs, is unsightly, and may not be sufficient to achieve tolerable dilutions of smells and other pollutants. Dilution can also be achieved by increasing the flow rate of the exhaust gas, but this method may also be inefficient and may be associated with costs for air conditioning. For both approaches, the energy costs related to moving and conditioning large amounts of air are significant. Dilution also does not prevent the actual emission of pollution, as pollution concentrations are simply lowered below sensory or regulatory thresholds.

Cleaning of emissions is another approach to lower odorous compounds and other pollutants. Bio-filters utilizing microorganisms are able to degrade organic and inorganic compounds in exhaust gas. However, they may be inefficient at high concentrations of pollutants and require maintenance by trained staff to maintain optimal pH, temperature and humidity conditions for microbial growth. The pollution stream feeding the bacteria has to be constant and large fluctuations can lead to death of the culture. Bio-filters are also prone to clogging and are of large size.

Chemical scrubbing of exhaust gas is another approach associated with high operation costs due to expenses for consumable chemical reactants, potential chemical hazards, and disposal of polluted water.

Yet another approach is adsorption of pollutants onto a solid, typically activated carbon or charcoal. This method is especially suited for capture of high molecular weight compounds present at a low concentration in the exhaust, but the efficiency deteriorates over time and the method also generates toxic waste, which needs to be disposed of in a proper manner. Charcoal filters generate a pressure drop that has to be overcome by additional fan power increasing operational costs.

Thermal combustion of exhaust gas is an effective method for cleaning air, but frequently creates new pollutants such as NOₓ and is expensive due to the high energy demands inherent in heating air to 300-1400° C. When pollutant concentrations are below the combustion limit, natural gas is added as fuel, driving up costs and CO₂ emissions, and therefore increasing the environmental footprint substantially.

Electrostatic precipitation is a well-known method for removing particles from air. It relies on inducing an electrical charge on particles and then attracting them in an electric field and separating them from the air stream. Disadvantages to the use of electrostatic precipitation include the expensive disposal of precipitated matter, failure to remove all particles in the smaller size range, and vulnerability to arcing in heavily polluted or wet airstreams. The method does not treat gas phase pollution.

Treatment of polluted air such as exhaust gas with ozone (i.e. ozonolysis) is another method that has been attempted. While it is efficient at removing some odors, many species do not react easily with ozone. Unfortunately, some of the chemical products of ozonolysis might be more hazardous than the initial compounds. Catalytic ozone oxidation of benzene has been described eg by Park et al. (Nonoscale Research Letters, 2012, 7:14, pages 1-5). The oxidation is carried out at low temperature and in the presence of MnOx/Al-SBA-16 catalyst. Other authors describe use of other specific catalysts such as manganese oxide, titanium dioxide or the like.

DE 102005 035951 (Nonnenmacher) teaches a method and apparatus for cleaning air, wherein the air to be cleaning is dosed with ozone, led through a moistened silica gel and subjected to UV light to generate hydroxyl radicals from the ozone.

US 2003/143140 (Hwang) teaches a method for removing nitrogen oxides, sulfur oxides, mercury, and mercuric oxide from gas streams from furnaces and other flue gas streams, wherein the gas stream is contacted with ozone to oxidize one or more of said compounds. The gas stream may be subject to scrubber treatment and/or exposed to UV-light.

DE 10 2010 017614 (NT Ablufttechnik) teaches a method wherein air to be treated is heated to above 100° C., dosed with ozone and subjected to an a catalyst, whereby ozone and dioxygen contained in the air to be treated reacts with pollutants in the air. The catalyst used is taught to be manganese oxide and copper oxide.

EP 2 072 110 (Arn) teaches a method for removal of waste gasses from composting, wherein the air to be treated is made very moist by spraying into it alkaline water, and mixing in peroxide and ozone, so that the pollutants react with ozone and peroxide within droplets.

### DETAILED DESCRIPTION OF THE INVENTION

The inventors of the present invention have found that reactive oxygen species formed by thermal decomposition of ozone are efficient at cleaning air streams containing odorous compounds and other pollutants such as exhaust gas. Such an exhaust gas cleaning system operates in a cheap and fast manner. No specific catalyst needs to be used such as eg MnOx/Al-SBA-16, titanium dioxide, mixtures of manganese oxide with eg aluminum oxide, ferri oxide, copper oxide, Pt with aluminum oxide and silicium oxide or the like.

The extent of applicability of the present invention is apparent from the detailed description below, but it should be understood that the specific examples as well as detailed description are included merely to illustrate the preferred embodiments, and that modification and alterations within the scope of protection will be obvious to persons skilled in the art on the basis of the detailed description.

The present invention relates to a method for cleaning a polluted airstream such as exhaust gas using thermal breakdown products of ozone. The exhaust gas to be cleaned and ozone is passed over a collision surface at a temperature sufficient to cause formation of said breakdown products and oxidation of polluting compounds in the gasstream by said breakdown products. The inventors have found that the rate of ozone decomposition at a collision surface is temperature dependent and that the rate abruptly increases above 47° C. Cheap and efficient methods or apparatuses for generating ozone may be employed. Details are specified here only to provide examples to aid a thorough understanding of the present invention. It will be apparent to a person skilled in the art, however, that the present invention may be practiced in other ways than demonstrated in the provided exemplary embodiments.

As mentioned above, the present inventors have observed that polluted gas can be cleaned in an environment containing heated ozone. The exact mechanism is presently not known; it may be that the heated ozone by contact with a collision surface (in this context also denoted “reaction surface”) forms reactive oxygen species that are responsible for the cleaning of the polluted gas. It may also be that the reaction to reactive oxygen species take place in the gas phase or another mechanism may be operating, or any a combination thereof. Thus, in the present context, the terms “on a collion surface”, “at a collision surface”, “over a collision surface” illustrate that the mechanism is not known, but the reactive oxygen species are formed in the vicinity of or after contact with a collision surface. In the priority application the term “reaction surface” was used, and the skilled person will understand that the terms “reaction surface” and “collision surface” can be used interchangeably to denote a surface that does not partake in the breakdown of ozone other than providing a surface for heated ozone to collide with. This means that whereas ozone shortly contacts the collision surface, the breakdown of ozone does not depend on chemisorption of ozone onto the collision surface, and no temporary intermediate is formed between ozone and the collision surface. Accordingly, the terms are “reaction surface” and “collision surface” used interchangeably. An essential observation is that no oxidation catalyst is involved in the cleaning process, i.e. neither in the surrounding gas nor in or on the reaction or collision surface. In general, oxidation catalysts include vanadium pentoxide, vanadium phosphate, platinum, mixed silver oxides, cobalt salts, manganese salts, molybdenium oxides, bismuth oxides, ferri oxides, mixed oxides eg of bismuth-molybdenium oxides or ferri-molybdenium oxides and the like. No catalysts are provided in the present invention, including in relation to the collision surface. The breakdown of ozone in the invention is thought to be due to heated ozone colliding with the collision surface, without interacting with the surface in any other way.

As mentioned above, the basis of the invention is to provide a method for cleaning gas containing one or more pollutants, the method being to expose the gas to thermal decomposition products of ozone. Ozone is brought into contact with a warm collision surface and the reaction preferably takes place at a temperature at 40° C. or more. The thermal breakdown is preferably taking place within a temperature range between 40-200° C., such as e.g. 40-150° C.; 40-130° C.; 40-120° C.; 45-110° C.; 45-105° C. or 45-100° C. The required temperature may be provided i) by the polluted gas, ii) by heating of the polluted gas before contact with ozone, iii) by heating of ozone before contact with the collision surface, iv) by heating the collision surface. Moreover, there may be more than one collision surface such as eg 2, 3, 4 or more to ensure sufficient reaction time with the collision surface(s). It is imagined that the collision surface(s) may be made of any material not consumed by the polluted gas or by ozone or ozone decomposition products. Specific examples are given herein. A requirement is that when ozone is heated, eg by bringing it in contact with a heated collision surface, and the ozone degradation products may be formed as a surface reaction of by a reaction in the vicinity of the collision surface. Then thermal decomposition products of ozone are formed that can interact with the polluted gas. Thermal decomposition products of ozone that are used to clean the polluted gas are also denoted reactive oxygen species.

In some cases, the cleaning process according to the present invention may be combined with other methods such as e.g. a scrubber. Thus, once the polluted gas has been subject to the method of the invention it may be led through a scrubber to remove residual pollutants.

### Possible Applications

One application of the present invention is to clean the exhaust gas of a biogas plant, but a person skilled in the art will know that the method may equally be used in other installations, such as without limitation, the exhaust gasses of forges or chemical plants, the air outlets of livestock production facilities or air inlets in buildings. The novel and inventive technology of the present invention is also amenable to installation in a small unit to clean air in a room or an office, a train, an airplane or any other confined space where the access to clean fresh air free of pollutants and odors is limited. This small unit may or may not be portable.

In one embodiment of the present invention, the method is used to clean polluted or odorous air such as exhaust gas resulting from biogas production. The device may be installed in a chimney, an exhaust outlet, or in a heating, ventilating, and air conditioning (HVAC) system. Means adapted to ensure airtight fit of the system to existing air stream carriers, ducts, stacks, or chimneys will be known to a person skilled in the art.

The term “exhaust gas” refers to a stream of gas that is produced by manufacturing, livestock production, combustion of biofuel or other fuels, chemical plants, forges, and so on, and is not limited to gases resulting from combustion. Thus, gaseous emissions that may comprise noxious or odious compounds and/or other pollutants is considered exhaust gas within the scope of this invention, as is atmospheric air which is polluted due to manufacturing, combustion, forging, or livestock production or other production. Emissions that are discharged from site of production or site of release by ventilation are also considered exhaust gas. Thus, terms such as “exhaust gas”, “polluted air”, “air stream” as using within this application all refer to gas having pollutants to be cleaned, wherein the gas may be a mixture of gasses such as atmospheric air.

### DESCRIPTION OF DRAWINGS

The invention is explained in detail below with reference embodiments referring to the Figures In FIGS. 1 and 2, the exhaust gas to be treated is sufficiently hot to allow breakdown of ozone, i.e. no further heating is necessary. In FIGS. 3 and 4, the exhaust gas is not sufficiently hot to initiate breakdown of ozone and, accordingly, heating elements are included to heat the exhaust air before the exhaust gas is contacted with ozone (or degradation products of ozone). In FIGS. 5 and 6, the exhaust gas to be treated is not sufficiently hot to cause breakdown of ozone. The heating elements are incorporated into the collision surface, and ozone is provided to the collision surface. Thus, both the heating and the breakdown of ozone may take place at the collision surface, where the reaction of the pollutant with the breakdown product(s) also may take place. In FIG. 7, the exhaust air is not sufficiently hot to breakdown ozone, but it is heated by means of a heat exchanger placed before the exhaust gas comes into contact with the collision surface or a vicinity of the collision surface. The cooling side of the heat exchanger is placed after the collision surface and allows to regain some of the energy needed to heat the gasstream in first place.

In FIG. 1, the exhaust gas to be treated (11) is hot exhaust gas, which is led into the device through the inlet (12) and is conveyed through the device as a continuous flow. Ozone (13) is infused into the hot gasstream (11) before the gasstream is passed over the collision surface, which in FIG. 1 is shown as a ring (14). This causes breakdown of ozone and formation of reactive species that will oxidize polluting compounds in the exhaust airstream. The collision surface can also be a grid, a honey-comb structure or any other form that ensures that most or all ozone reacts on or near the surface while ensuring that the pressure drop is small. The retention time in the device is sufficient to warrant breakdown of polluting compounds in the gasstream. The treated gas exits the device through the outlet (15) and can be released into the indoor or outdoor environment directly or through e.g. a chimney, or a ventilation system. Preferably ozone is infused immediately after a heating or combustion event, such that sufficient heat is retained in the gas to be treated.

In FIG. 1, the numbers relate to:
11: hot airflow of exhaust gas
12: inlet
13: ozone
14: collision surface
15: outlet

In FIG. 2, the exhaust gas to be treated (21) is hot exhaust gas, which is led into the device through the inlet (22) and is conveyed through the device as a continuous flow. Ozone (23) is infused into the hot gasstream from the collision surface (24), typically through one or more openings in said surface (25) and is broken down to yield reactive species that oxidize polluting compounds in the gasstream. The collision surface can also be a grid, a honey-comb structure or any other form from which ozone is infused, that ensures that most or all ozone reacts on or near the surface while ensuring that the pressure drop is small. The treated gas exits the device through the outlet (26) and can be released into the indoor or outdoor environment directly or through e.g. a chimney, or a ventilation system.

In FIG. 2, the numbers relate to:
21: hot gasflow
22: inlet
23: ozone
24: collision surface with ozone infusion
25: holes
26: outlet

In FIG. 3, the gasflow to be treated (31) is not sufficiently hot to induce breakdown of ozone on the collision surface. The gas is led into the device through the inlet (32) and is conveyed through the device as a continuous flow and is heated by one or more heating elements (33) to the required temperature, before ozone (34) is injected into the gasstream, and before the resulting mix passes over the collision surface (35) to yield reactive ozone breakdown species capable of oxidizing polluting compounds in the gasstream. The collision surface can also be a grid, a honey-comb structure or any other form that ensures that most or all ozone reacts on or near the surface while ensuring that the pressure drop is small. The treated gasflow exits the device through the outlet (36). The outlet may lead into e.g. a chimney, a ventilation system, or may be just released directly into the indoor or outdoor environment.

In FIG. 3, the numbers relate to:
31: gasflow
32: inlet
33: heating element(s)
34: ozone
35: collision surface
36: outlet

In FIG. 4, the gasflow to be treated (41) is not sufficiently hot to warrant breakdown of ozone on the collision surface. The gasflow is led into the device through the inlet (42) and is conveyed through the device as a continuous flow. In the device the gas is heated sufficiently by one or more heating elements (43) before the gasstream passes over the collision surface (44) with openings (45) from which ozone (46) is infused to yield reactive ozone breakdown species to oxidize polluting compounds in the gasstream. The collision surface can also be a grid, a honey-comb structure or any other form that ensures that most or all ozone reacts on or near the surface while ensuring that the pressure drop is small. The treated gasflow exits the device through the outlet (47). The outlet may lead into e.g. a chimney, a ventilation system, or may be just released directly into the indoor or outdoor environment.

In FIG. 4, the numbers relate to:
41: gasflow
42: inlet
43: heating element(s)
44: collision surface with ozone infusion
45: holes
46: ozone
47: outlet

In FIG. 5, the gasflow (51) to be treated is not sufficiently hot to warrant breakdown of ozone on the collision surface. The gasflow is led into the device through the inlet (52) and is conveyed through the device as a continuous gasflow. Ozone (53) is infused into the gasflow, which is then heated sufficiently by heating elements incorporated in the collision surface (54) to yield reactive ozone breakdown species to oxidize polluting compounds in the gasstream. The heating element and collision surface can also be a grid, a honey-comb structure or any other form that ensures that most or all ozone reacts on or near the surface while ensuring that the pressure drop is small The treated gasflow exits the device through the outlet (55). The outlet may lead into e.g. a chimney, a ventilation system, or may be just released directly into the indoor or outdoor environment.

FIG. 5
51: gasflow
52: inlet
53: ozone
54: collision surface with heating element(s)
55: outlet

In FIG. 6, the gasflow (61) to be treated is not sufficiently hot to warrant breakdown of ozone on the collision surface. The gasflow is led into the device through the inlet (62) and is conveyed through the device as a continuous gasflow. The gasflow is thus heated sufficiently by heating elements incorporated in the collision surface (63) from which ozone (64) is infused via holes (65) to yield reactive ozone breakdown species to oxidize polluting compounds in the gasstream. The collision surface form which ozone is infused can also be a grid, a honey-comb structure or any other form that ensures that most or all ozone reacts on the surface while ensuring that the pressure drop is small The treated gasflow exits the device through the outlet (66). The outlet may lead into e.g. a chimney, a ventilation system, or may be just released directly into the indoor or outdoor environment.

In FIG. 6, the numbers refer to:
61: gasflow
62: inlet
63: collision surface
64: ozone
65: holes
66: outlet

In FIG. 7, the gasflow (71) to be treated is not sufficiently hot to warrant breakdown of ozone on the collision surface. The gasstream is led into the device through the inlet (72) and is conveyed through the device as a continuous stream. In this embodiment, the gasflow is heated sufficiently by the heating side (73) of a heat exchanger (74), before passing over the collision surface (75) from which ozone (76) is infused via holes (77) to yield reactive ozone breakdown species to oxidize polluting compounds in the gasstream. The collision surface from which ozone is infused can also be a grid, a honey-comb structure or any other form that ensures that most or all ozone reacts on the surface while ensuring that the pressure drop is small. The treated gas is passed through the cooling side (78) of said heat exchanger (74) to harvest heat which is then used to heat the ingoing gasflow. For the sake of simplicity FIG. 7 depicts a configuration where ozone (76) is infused from the collision surface (75), but any of the mentioned configurations of ozone infusion or injection will be applicable. The treated gasflow exits the device through the outlet (79). The outlet may lead into e.g. a chimney, a ventilation system, or may be just released directly into the indoor or outdoor environment.

In FIG. 7, the numbers relate to:
71: gasflow
72: inlet
73: heat exchanger (heating side)
74: heat exchanger
75: collision surface with ozone infusion
76: ozone
77: holes
78: heat exchanger (cooling side)
79: outlet

In FIG. 8, the exhaust gas to be treated (81) is hot exhaust gas, which is led into the device through the inlet (82) and is conveyed through the device as a continuous flow. Ozone (83) is infused into the hot gasstream (81) before the gasstream is passed over the collision surface, which in FIG. 8 is shown as a ring (84). This causes breakdown of ozone and formation of reactive species that will oxidize polluting compounds in the exhaust gasstream. The collision surface can also be a grid, a honey-comb structure or any other form that ensures that most or all ozone reacts on the surface while ensuring that the pressure drop is small. The retention time in the device is sufficient to warrant breakdown of polluting compounds in the gasstream. Other potential pollutants that entered the device or that were created during the oxidation processes are then removed in a second treatment stage (85), that could be but is not limited to: a (wet) scrubber or a catalytic converter. The treated gas exits the device through the outlet (86) and can be released into the indoor or outdoor environment directly or through e.g. a chimney, or a ventilation system. Preferably ozone is infused immediately after a heating or combustion event, such that sufficient heat is retained in the gas to be treated. For the sake of simplicity FIG. 8 depicts the second treatment stage following a configuration similar to FIG. 1. However, it should be pointed out that a second treatment stage could follow any of the other configurations (FIG. 2-7).

In FIG. 8, the numbers relate to:
81: hot gasflow of exhaust gas
82: inlet
83: ozone
84: collision surface
85: second treatment stage
86: outlet

FIG. 9 shows the setup used in the examples herein. The exhaust gas and ozone is led through an oven via a very small tube, which is the collision surface. The dimensions of the tube ensure that ozone comes into contact with the surface (or into the vicinity of the collision surface) so as to breakdown ozone to the reactive species. As seen from the examples herein, a tube of stainless steel as well as a tube of Teflon has been used successfully as collision surfaces.

FIG. 10 shows the relationship between gas temperature and oven temperature in the setup shown in FIG. 8. To achieve an gas temperature of about 45-50° C., the temperature of the oven must be from about 100° C. to about 130° C. If another setup is used, a person skilled in the art will know how to use a heating element in order to increase gas temperature to obtain the best conditions for breakdown of ozone.

FIG. 11 shows that the gas temperature must be above 40° C. in the experimental setup shown in FIG. 9 in order to yield a certain degree of efficiency. It is contemplated that the ozone removal efficiency (i.e. the ability of ozone to remove pollutant) is dependent on the temperature used to convert ozone to the reactive oxygen species. The collision surface is also important as the heated ozone is converted to the reactive oxygen species by collision of ozone to the collision surface, but FIG. 11 shows that the temperature for the conversion is relatively independent on the material used in the collision surface, at least regarding to stainless steel and Teflon.

FIG. 12 shows the ozone removal efficiency dependent on the surface area of the collision surface. It clearly shows that a larger surface area increases the ozone removal efficiency. FIG. 12 also indicates that for each setup used it is important to investigate the relationship between oven/heating element temperature and gas temperature, gas temperature and ozone removal efficiency, and gas temperature and surface area of the collision surface employed.

FIG. 13 is a graph showing that the volume of the tube used as collision surface in the setup shown in FIG. 9 has no or only little influence on the ozone removal efficiency. As shown in FIG. 12, however, the surface area is of importance.

FIG. 14 is an extended version of the setup in FIG. 9 including addition of testpollutant H₂S to the exhaust gas and ozone. The output of sulfur containing species is monitored as well.

FIG. 15 shows the efficiency of removing H₂S in the setup illustrated in FIG. 14 when low ozone concentration is used (a) or when high ozone concentration is used (b). The higher ozone concentration the better the efficiency.

By ozone generator is meant a stand-alone or built-in device that is capable of generating ozone which can then be used in the present invention. Typically ozone generators are able to generate ozone from atmospheric oxygen and do not require separate oxygen input. Several methods are available for the generation of ozone, for example by corona discharge; by ultraviolet light; by electrolytic ozone generation wherein H₂O is split into H₂, O₂ and O₃; or by cold plasma. It is preferred to use a method wherein oxygen from atmospheric gas or from the exhaust gas to be treated is used. Ozone generators are commercially available.

As seen from the above, heating of ozone to a suitable temperature may be effected by i) employment of heated exhaust gas, ii) heating elements provided upstream of the collision surface (either to heat exhaust gas or a mixture of exhaust gas and ozone), iii) heating elements incorporated in the collision surface, or by use of a heat exchanger.

In order to enable the breakdown of ozone, the temperature of ozone (or the collision surface) should exceed 40° C. In those cases, where external heating is provided, the external heating should provide a temperature of from about 40° C. to about 200° C. or from about 45° C. to about 150° C. As demonstrated herein, a suitable temperature may be determined by investigating the ozone removal efficiency dependent on the gas temperature, the gas temperature dependent on the temperature of the oven (or the heating elements), and the ozone removal efficiency dependent on surface area of the collision surface employed. Based on the guidance given in the examples herein, a person skilled in the art will know how to determine relevant parameters.

When used, the heat exchanger may also be placed downstream of the collision surface in order to recover heat from the exhaust. The heat can be used for heating the incoming gasflow or the heat energy can be reused for any other means. Methods for recovering heat energy will be known to a person skilled in the art.

Heating of gas or ozone is achieved in the examples in this application by passing the gas through pipes placed in an oven. Heating of gas can be achieved by other means as well, including by passing gas over heating elements such as heating coils, or by passing gas through a heat exchanger. Importantly, the exhaust gas to be treated may not need further heating but is already hot enough from e.g. combustion or other upstream processing.

The collision surface used may be in any suitable form. It may be in the form of a narrow tube through which the exhaust gas and ozone pass, or it may be in the form of a grid with openings sufficiently small for interaction between the ozone and the collision surface to take place and at the same time sufficiently large to avoid a build-up of undesired pressure. The collision surface may also be in the form of many plates placed in a stack, where the plates have openings, but the plate beneath one plate has openings where the upper plate does not have any opening. Thus, the flow through such an arrangement ensures that ozone is brought into contact with a collision surface. As mentioned above, the collision surface may be heated by a heating element, or the heating of the ozone may be provided by other means. Importantly, as mentioned before, the collision surface does not contain any oxidation catalysts such as those mentioned herein. Especially, the collision surface has not been coated with such an oxidation catalyst.

By collision surface is meant any surface on which/at which a chemical reaction can take place. In a preferred embodiment the collision surface is inert (ie in the meaning that it does not contain any oxidation catalysts and thus does not form a temporary intermediate with the ozone) and can withstand reactive species in exhaust gas as well as ozone. However, as mentioned herein before, when ozone is contacted with or ozone is in the vicinity of the surface reactive oxygen species are formed. Thus, it may be stainless steel, Teflon (i.e. polytetrafluoroethylene, PTFE), glass, Kynar (polyvinylidene fluoride resin, PVDF), CPVC, Lexan (polycarbonate resin), Hypalon (chlorosulfonated polyethylene (CSPE) synthetic rubber (CSM)), PCTFE (polychlorotrifluoroethylene), PVC (polyvinylchloride), EPDM, Viton (synthetic rubber and fluoropolymer elastomer), or another inert material and may be crafted typically as a permeable membrane, beads, a honeycomb structure, or a grid, ensuring optimal decomposition of ozone. The pressure drop across the surface is preferably small, such as e.g. 100 Pa.

The term gas refers to atmospheric air or other mixtures of gasses, typically exhaust gasses; indoor air to be recycled, or outdoor air to be cleaned before it is released indoors, which may contain pollutants such as H₂S, CH₃SH, tetrahydrofuran, toluene or other odorous and or pollutant compounds or combinations thereof.

In all embodiments, a scrubber may be placed downstream of the collision surface to remove soluble oxidation products, e.g. H₂SO₄ and SO₂, cf. FIG. 8. Scrubbers are well known in the art and a person skilled in the art will know how to incorporate a scrubber.

Excess ozone not consumed in the reaction will be dealt with with knowledge of those skilled in the art. This includes repeated treatment stages using the described invention, if for example a single pass over a collision surface is not sufficient in yielding the desired removal. Multiple instances of the described invention can be based on any of the suggested designs (FIGS. 1-8).

These specific embodiments in the foregoing in no manner exhaust the applicability of the present invention, and it will be evident to a person skilled in the art that various modifications and changes may be made to the invention without departing from the broader scope of the invention. The present application will be described in further detail by the following non-limiting examples.

The examples below relate to the breakdown of H₂S, but the invention is expected to be able to break down any pollutant susceptible to degradation by ozone decomposition products. Thus, the invention can be used to clean exhaust air or gasses, recycled indoor air, or outdoor air before its release indoor.

### EXAMPLES

### Materials:

ACF-1000 Ozone generator (0₃ Technology AB)

### Oven (284 52 C, Elektro Helios)

Mercury thermometer (temperature range: 20-240° C. (built-in in oven) or 20-200° C. (in air outlet)
Variable area flow meters (model FLDA3326G (0-1 L/min), FLDA3215ST (0-10 L/min) Omega)
UV-100 ozone monitors (ECO SENSORS)
Stainless steel tube 3R60 SS 2353-22, Sandvik
Teflon tube PFA-T4-047-100, Swagelok
H₂S (Yara Praxair, 100 ppm (in N₂)
Critical flow orifice: 88⅛″-×-⅛″-NPT-CAL-100 (Lenox Laser)
Sulfur monitor 450i (Thermo Scientific)

### Technical air

### Example 1: The Production of Reactive Oxygen Species Using a Heated Surface

This example demonstrates the production of reactive oxygen species in an gas stream by passing ozone-enriched gas over a heated surface.

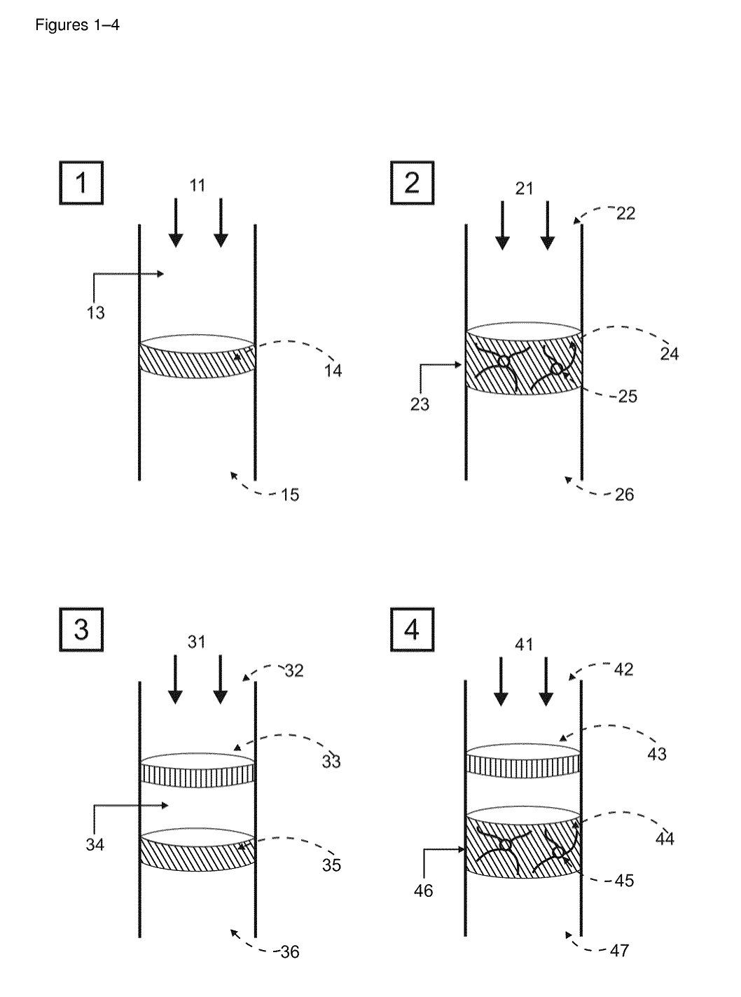

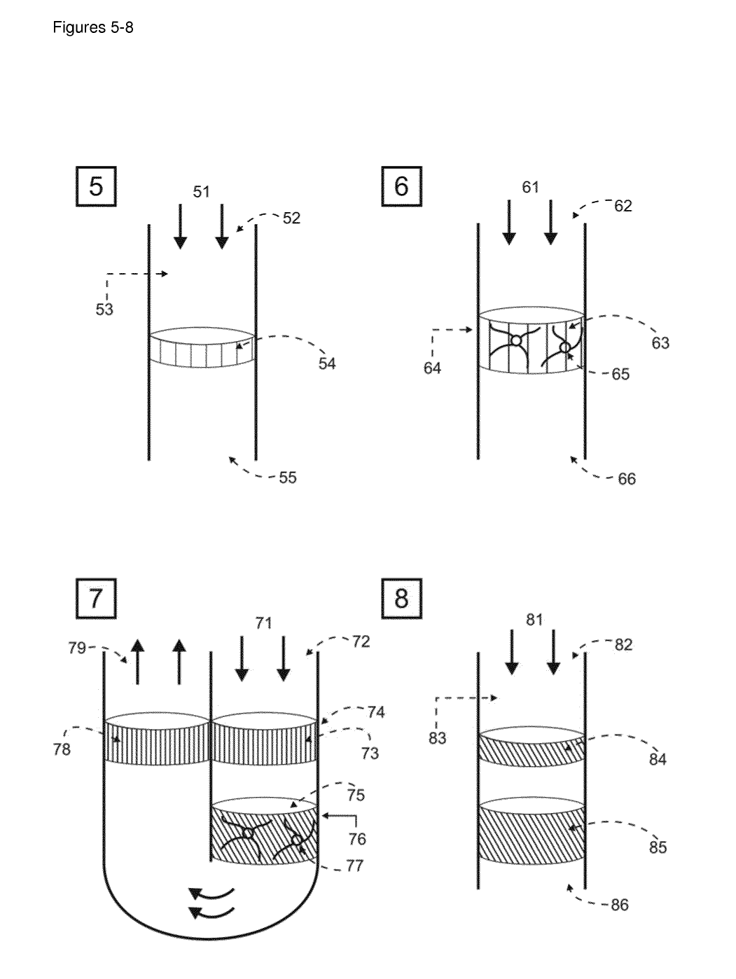

The experimental system (FIG. 9) consists of an inlet for technical air, an air flow splitter, an inline ozone generator which can be bypassed, an oven in which tubing made from different materials and of various geometries can be placed, and a thermometer in the oven air outlet. The oven temperature is measured by an internal thermometer. Air is lead into the system and separated into two flows, one leading into the ozone generator and one bypassing it, in order to regulate the amount of ozone produced. The flow rate of air into the ozone generator is controlled by a flow meter, and the flow rate of bypassing air is also controlled by a flow meter.

The flow rate in this example was 3 L/m in the oven tubes.

The air stream exiting the ozone generator was mixed with the air bypassing the ozone generator to obtain a controlled ozone concentration in this example of 6.59±0.79 ppm or 17.45±1.43 ppm, which was measured with an ozone monitor. The mixed air was then lead into either a stainless steel tube or a Teflon tube located inside the oven. Tube dimensions and O₃ concentrations are shown in table 1.

As the tubes are located inside the oven the air stream is heated, and the inner surface of the tubing functions as a collision surface leading to decomposition of ozone. The retention time in the stainless steel tube in the oven is approximately 0.4 seconds and the retention time in the Teflon tube in the oven is approximately 0.61 seconds. The outlet of the tubing in the oven is equipped with a thermometer to measure the exiting air temperature. The airstream is lead into a cooling pipe of 2.8 m to permit measurement of ozone in an ozone monitor functioning in the range 10-40° C., cf. FIG. 9.

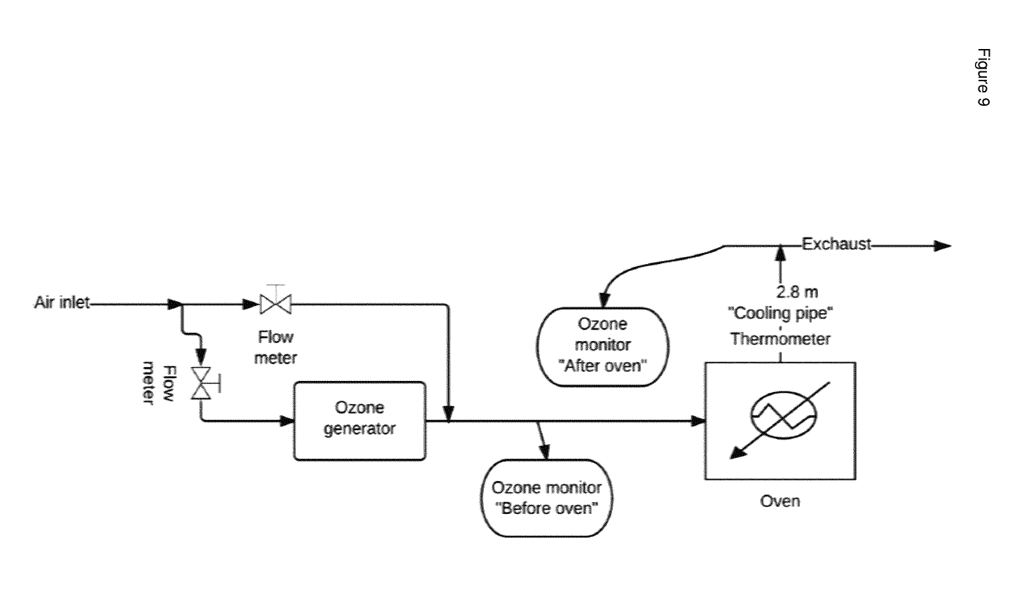

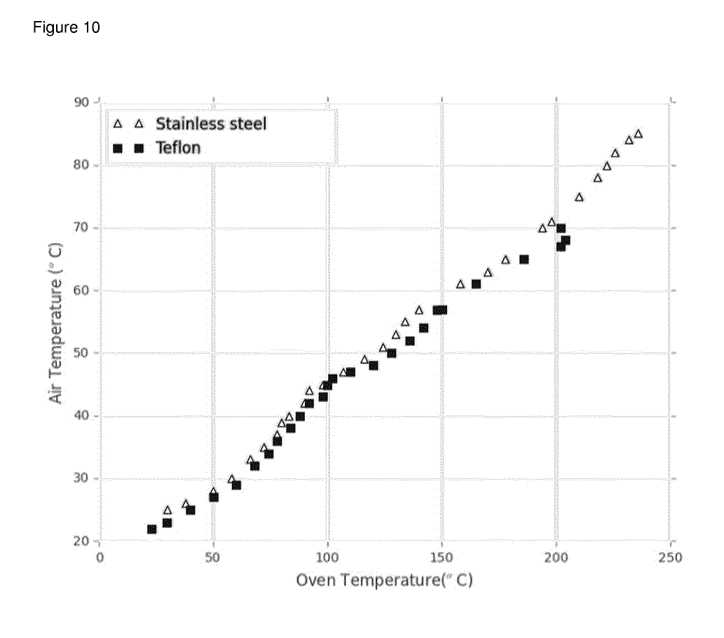

By adjusting the oven temperature from 20° C. to 180° C., a defined exiting air temperature between 20-90° C. can be achieved. In this setup the air temperature was found to be linearly dependent of oven temperature (see FIG. 10).

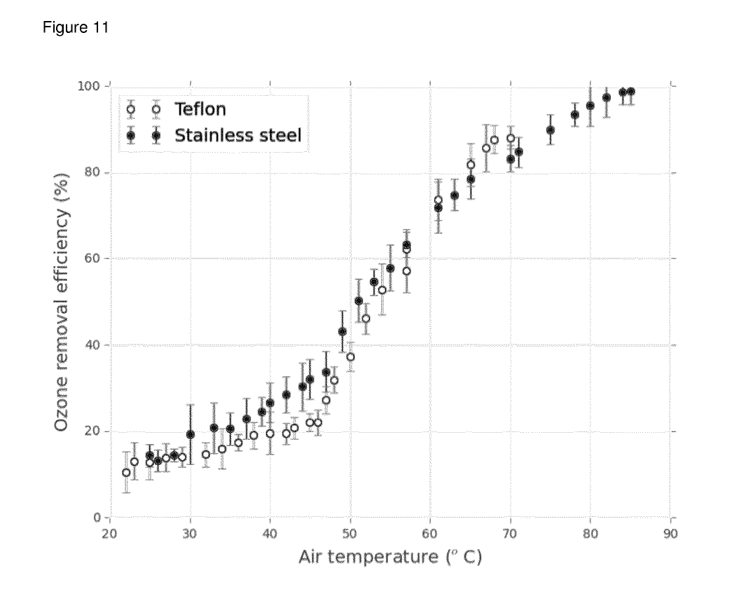

Reduced ozone content as a measure of ozone decomposition was found to depend on the temperature of the air exiting the oven, such that in the temperature range 20-47° C., 15-35 percent of ozone was removed, whereas in the temperature range 47-85° C. 35-99±3 percent of ozone was removed. Increasing exit air temperature results in reduced measured ozone content, i.e. greater ozone decomposition (see FIG. 11). Around an air temperature of 85° C. nearly all ozone was decomposed under the employed experimental conditions.

### Example 2: The Production of Decomposed Ozone is Dependent on the Area of the Heated Surface

This example demonstrates that the decomposition of ozone in an gas stream by passing ozone-enriched air over a heated surface is increased with larger surface area, not larger air reaction volume.

Using a modified version of the apparatus of Example 1, the tubing in the oven was stainless steel. Sets of stainless steel tubing (or stainless steel with additional Teflon tubing for mounting in oven) were interchanged to allow for comparison between constant volume and constant surface area of the tube (see table 1). As the tubes are located inside the oven the air stream is heated, and the inner surface of the tubing functions as a collision surface on which ozone is decomposed.

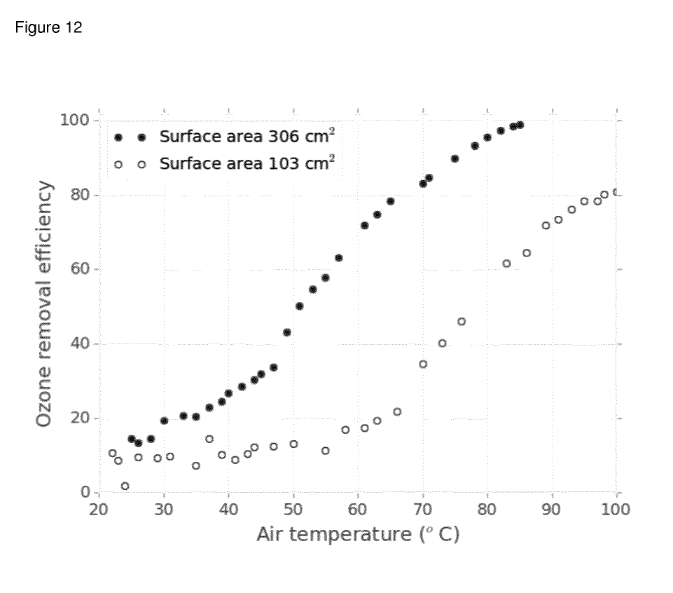

Ozone removal was found to depend on surface area and not on volume of the stainless steel tube, such that at an air temperature of 85° C., 100 percent of ozone was decomposed in a tube with a surface area of 306 cm², compared to approximately 60 percent removal with a surface area of 103 cm², both tubes having a volume of approximately 20 cm³ and retention times of 0.37 to 0.4 seconds (see FIG. 12 and table 1).

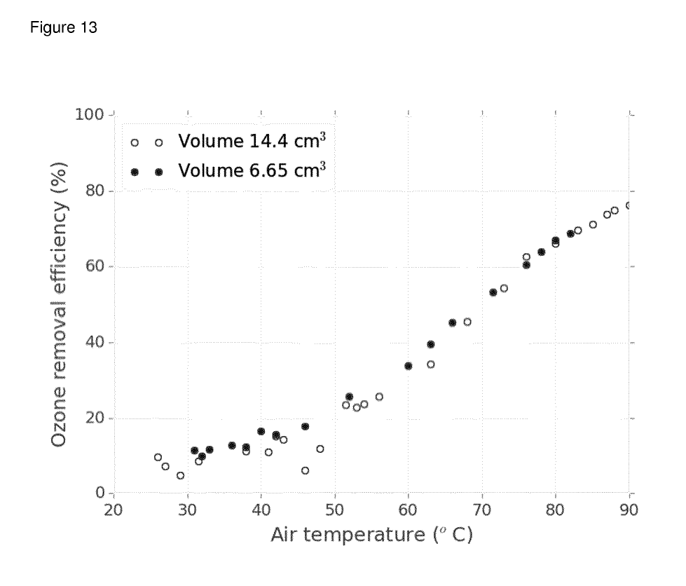

When surface area was kept at 80 cm² there was no difference in ozone removal between a tube volume of 6.65 cm³ and a tube volume of 14.4 cm³. Retention time varied between 0.15 sec and 0.32 sec (see FIG. 13).

Thus, the activation of ozone depends on the temperature and the surface area, and it is possible to obtain complete decomposition of ozone in this system.

### Example 3: The Use of Decomposed Ozone to Degrade Hydrogen Sulfide (H₂S)

This example demonstrates the production of decomposed ozone in an gas stream by passing ozone-enriched air over a heated surface and its use to degrade hydrogen sulfide (H₂S) present in the air stream.

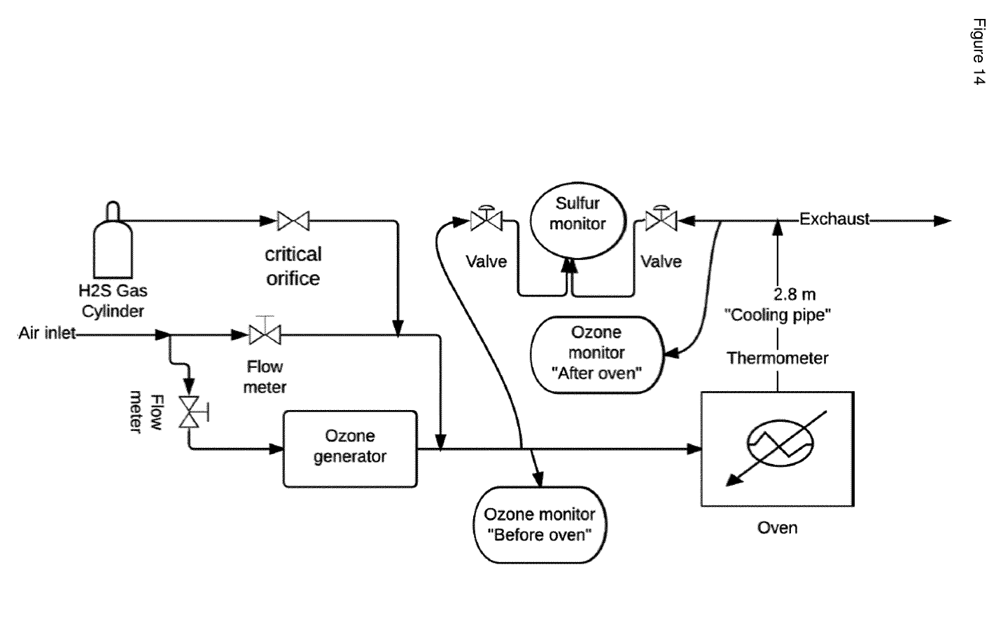

This example uses a modified version of the technical system described in Example 1, in that H₂S can be injected through a critical flow orifice into the air stream bypassing the ozone generator before the air streams are mixed. From the mixed air stream sample air can be diverted to a sulfur monitor, as can cooled sample air from the post-oven cooling pipe (see FIG. 14 for experimental setup).

In this setup the stainless steel tube in the oven has a volume of 20 cm³ and a surface area of 332.2 cm².

In this example the air flow into the system was approximately 4 L/min and concentration of H₂S in the pre-oven airstream was 3.91 ppm. This experiment was carried out using two concentrations of ozone (6.59±0.79 ppm or 17.45±1.43 ppm). The temperature of the oven was 189±7° C. and the exiting air temperature was 80±4° C.

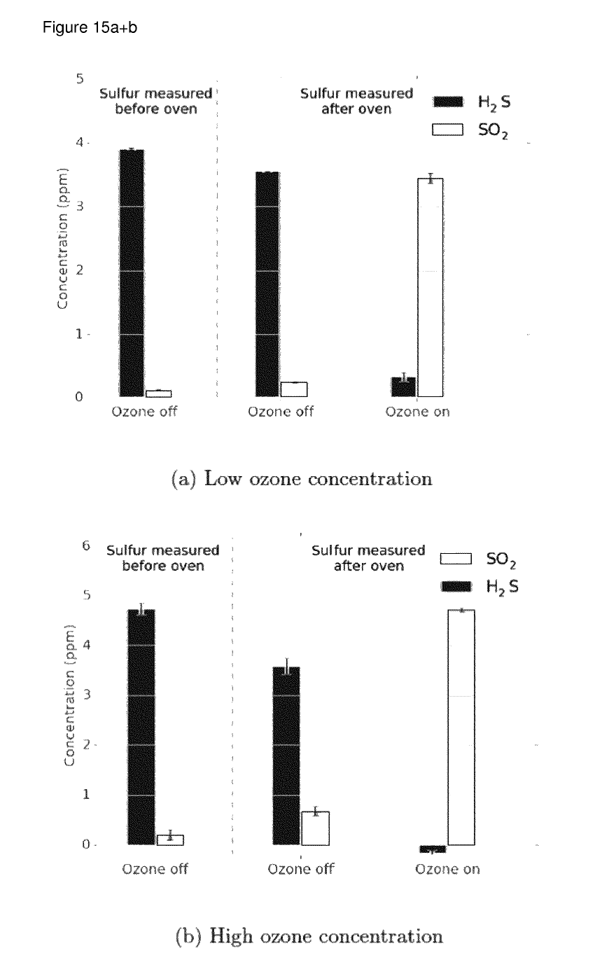

The low concentration of ozone caused a 90 percent reduction of H₂S (from 3.91 ppm to 0.33 ppm), whereas the high concentration of ozone caused a complete removal of H₂S (see FIGS. 15a and 15b).

Thus, reactive oxygen species are efficient in oxidizing H₂S in an airstream. The inventors find that the invention will also be able to oxidize a vast number of other pollutants, such as CH₃SH, DMS, CS₂, tetrahydrofuran, toluene, formaldehyde, NH₃.

In the examples above the oxidation product of H₂S is SO₂, likely due to the fact that dry technical air was used. In many applications sufficient amounts of H₂O will be present in the treated air, so that SO₃ and H₂O forms H₂SO₄ instead of SO₃ decomposing and forming SO₂.

TABLE 1

| | Same volume | | Same surface area | |
|---|---|---|---|---|
| | Tube diameter | | | |
| | 0.25 cm | 1 cm | 0.25 cm | 1 cm |
| Tube length | 390 (390) | 24.4 (45.4) | 43.3 (79.3) | 12.6 (48.6) |
| (cm) | | | | |
| Surface (cm²) | 306 | 76.7 (103) | 34.0 (79.2) | 39.6 (84.8) |
| Volume (cm³) | 19.1 | 19.2 (21.8) | 2.13 (6.65) | 9.9 (14.4) |
| Retention | 0.37 | 0.35 (0.40) | 0.047 (0.15) | 0.22 (0.32) |
| time (s) | | | | |
| [O₃]₀ “before | 65.8 ± 3.0 | 65.4 ± 4.9 | 61.7 ± 6.4 | 55.4 ± 3.6 |
| oven” (ppm) | | | | |

1. A method for cleaning exhaust gas comprising one or more pollutants, the method comprising contacting the gas with thermal decompositions products of ozone.

2. A method according to claim 1, wherein thermal decomposition products of ozone are obtained by contacting heated ozone with a collision surface.

3. A method according to claim 1, wherein thermal decomposition products of ozone are obtained by contacting ozone with a heated collision surface.

4. A method according to claim 1, wherein ozone is heated to a temperature of at least 40° C.

5. A method according to claim 1, wherein the thermal decomposition products of ozone are obtained by passing ozone over a heated collision surface at a temperature of at least 40° C.

6. A method according to claim 2, wherein the exhaust gas and the ozone are mixed before contact with or passing over the collision surface.

7. A method according to any of claim 2, wherein ozone is provided through said collision surface.

8. A method according to claim 1, wherein exhaust gas is heated before passing over the collision surface.

9. A method according to claim 2, wherein the collision surface is an inert surface.

10. A method according to claim 2, wherein the collision surface is a heat conducting surface.

11. A method according to claim 2, wherein the collision surface is stainless steel, Teflon (i.e. polytetrafluoroethylene, PTFE), glass, Kynar (polyvinylidene fluoride resin, PVDF), CPVC, Lexan (polycarbonate resin), Hypalon (chlorosulfonated polyethylene (CSPE) synthetic rubber (CSM)), PCTFE (polychlorotrifluoroethylene), PVC (polyvinylchloride), EPDM, Viton (synthetic rubber and fluoropolymer elastomer), or another inert material.

12. A gas cleaning device, comprising:
an inlet for exhaust gas comprising one or more pollutants;
an inlet for ozone;
optionally one or more heating elements;
a zone comprising a collision surface for decomposing ozone to reactive oxygen species;
a zone for reacting the reactive oxygen species with the one or more pollutants; and
a second treatment stage downstream of the collision surface to decompose any excess of ozone or a scrubber.

13. A gas cleaning device according to claim 12, further comprising a scrubber.

14. A gas cleaning device according to claim 12, further comprising one or more additional zones comprising a collision surface for decomposing ozone to reactive oxygen species.

15. A gas cleaning device, comprising:
an inlet for exhaust gas comprising one or more pollutants;
an inlet for ozone;
a zone comprising a collision surface for decomposing ozone to reactive oxygen species; and
a zone for reacting the reactive oxygen species.

16. A gas cleaning device according to claim 15, further comprising one or more heating elements.

17. A gas cleaning device according to claim 15, further comprising a second treatment stage downstream of the collision surface to decompose any excess of ozone or a scrubber

18. A gas cleaning device according to claim 15, wherein the collision surface is an inert surface.

19. A gas cleaning device according to claim 15, wherein the collision surface is a heat conducting surface.

20. A gas cleaning device according to claim 15, wherein ozone is heated to a temperature of at least 40° C.
